(19) United States
(12) Patent Application Publication
(10) Pub. No.: US 2020/0390468 A1
(43) Pub. Date: Dec. 17, 2020

(54) ABRASIVE SKIN CARE APPLIANCES

(71) Applicant: AGE SCIENCES, INC. dba PMD BEAUTY, Draper, UT (US)

(72) Inventor: Sam Alexander, Alpine, UT (US)

(73) Assignee: AGE SCIENCES, INC. dba PMD BEAUTY, Draper, UT (US)

(21) Appl. No.: 16/441,955

(22) Filed: Jun. 14, 2019

(51) Int. Cl. A61B 17/54

(52) U.S. Cl. CPC A61B 17/54; A45D 2200/207; A45D 2200/1054

(57) ABSTRACT

The present disclosure is drawn to an abrasive skin care appliance, which can include a facial surface having an abrasive matrix attached thereto, wherein the abrasive matrix comprises abrasive crystals having an average particle size from 25 μm to 250 μm and a plurality of metal-containing particles having an average particle size from 5 nm to 2 μm associated with surfaces of the abrasive crystals.

### BACKGROUND

Skin care is a rapidly evolving industry where treatment devices, skin care formulations, and related treatment protocols are being developed on nearly a continuous basis. Dermal treatment to promote or prolong the appearance of healthy skin, particularly facial skin, can be complicated for consumers, as there are many choices available and some approaches tend to work better than others for a given skin care goal and skin type. For example, skin cleansing and treatment for antimicrobial purposes (e.g., acne), may demand a different group of treatment protocols than skin tightening or microdermabrasion, and even within a narrower category of treatment, there can be many choices.

### BRIEF DESCRIPTION OF THE DRAWINGS

FIG. 1 illustrates a schematic view of an example microdermabrasion skin care device with multiple views of an example abrasive skin care appliance attached or attachable thereto in accordance with the present disclosure;

FIG. 2 illustrates schematic views of alternative example abrasive skin care appliances and associated example interchangeable vacuum chambers in accordance with the present disclosure;

FIG. 3 illustrates a schematic view of an example manual abrasive skin care appliance in accordance with the present disclosure;

FIG. 4 illustrates multiple views of an example vibratory skin care device with an example abrasive skin care appliance attached thereto in accordance with the present disclosure;

FIG. 5 illustrates a more detailed view of a side view of the example skin care device of FIG. 4 in accordance with the present disclosure; and

FIG. 6 provides various magnified images of example abrasive matrices applied to facial surfaces of abrasive skin care appliances in accordance with the present disclosure.

### DETAILED DESCRIPTION

The present disclosure is drawn to abrasive skin care appliances, which can be in the form of skin care devices, attachments for skin care devices, inserts for skin care devices, or the like. In one example, an abrasive skin care appliance can include a facial surface having an abrasive matrix attached thereto, wherein the abrasive matrix comprises abrasive crystals having an average particle size from 25 μm to 250 μm and a plurality of metal-containing particles having an average particle size from 5 nm to 2 μm associated with surfaces of the abrasive crystals. The abrasive crystals can include aluminum oxide particles, magnesium oxide particles, sodium chloride particles, sodium bicarbonate particles, or diamond particles, for example. In one example, the abrasive crystals can have an average particle size from 40 μm to 180 μm and/or the grit count of the abrasive matrix can be from 80 to 280. In one example, the metal-containing particles can have an average particle size 15 nm to 1 μm in some examples. The metal-containing particles can include gold particles or gold alloy particles, silver particles or silver alloy particles, copper particles or copper alloy particles, zinc particles or zinc alloy particles, platinum particles or platinum alloy particles, or a combination thereof. Alternatively, the metal-containing particles can be a metal salt of gold, silver, copper, zinc, and/or platinum. The abrasive crystals and the metal-containing particles can be present in the abrasive matrix at a weight ratio from 3:1 to about 500:1. In one example, the abrasive skin care appliance can be an insert or a pad shaped for associating with a microdermabrasion device, or the abrasive skin care appliance can be attached to a microdermabrasion device. In one example, the microdermabrasion device can be a rotational microdermabrasion device including a rotational motor to rotate the insert or pad and can further include a vacuum to remove skin debris away from the abrasive matrix. The rotational motor can be rotatable at one or more settings from 2,000 RPM to 7,000 RPM, from 3,000 RPM to 6,000 RPM, or from 3,400 RPM to 5,010 RPM. In another example, the microdermabrasion device can be a vibrational microdermabrasion device with a vibrational motor positioned within a head where the abrasive matrix contacts a skin surface. The vibrational motor can be oscillatable at one or more settings from 2,000 VPM to 12,000 VPM, or from 3,000 VPM to 6,000 VPM, for example. In some examples, the microdermabrasion device can include a secondary appliance associated therewith, such as a skin-cleansing appliance, a heating appliance, a cooling appliance, a massaging appliance (vibratory or unpowered), electromagnetic energy appliance, an ionic infusion appliance, a phototherapy appliance, a porous scrubbing appliance, or a combination thereof.

In another example, a method of making an abrasive skin care appliance can include combining abrasive crystals with metal-containing particles at a weight ratio from 3:1 to about 500:1 to form an abrasive particulate blend. The abrasive crystals can have an average particle size from 25 μm to about 250 μm and the metal-containing particles can have an average particle size from 5 nm to 2 μm. The method can further include softening a facial surface of an appliance substrate and pressing the facial surface into the abrasive particulate blend while the facial surface is in a softened state to form an abrasive matrix partially embedded in the facial surface. The abrasive crystals can include aluminum oxide particles, magnesium oxide particles, sodium chloride particles, sodium bicarbonate particles, or diamond particles, for example. The abrasive crystals can have an average particle size from 40 μm to 180 μm and/or the abrasive matrix can have a grit count of 80 to 280. The metal-containing particles can include gold particles or gold alloy particles, silver particles or silver alloy particles, copper particles or copper alloy particles, zinc particles or zinc alloy particles, platinum particles or platinum alloy particles, or a combination thereof. Alternatively, the metal-containing particles can be a metal salt of gold, silver, copper, zinc, and/or platinum. The facial surface of the appliance substrate can include a solvent-softenable plastic material wherein softening is by chemical softening. Chemical softening may include contacting the facial surface with a solvent selected from 1,2-dichloroethane, acetone, cyclohexanone, dichloromethane, methyl ethyl ketone (MEK), methyl benzene, tetrahydrofuran, or a combination thereof. The solvent-softenable plastic material can be selected from acrylic polymer, methacrylic polymer (e.g., polymethyl methacrylate), acrylonitrile butadiene styrene, polyacetal, cellulose acetate butyrate, polyethylene (e.g., LDPE, HDPE, UHDPE, PEX, etc.), polycarbonate, polyethylene terephthalate, polyethylene terephthalate glycol, polypropylene, polystyrene, polyvinyl chloride, or combinations thereof. The facial surface of the appliance substrate can alternatively (or additionally) be a thermally-softenable plastic material and softening can be carried out by thermal softening. The thermally-softenable plastic material can be a thermoplastic, such as one or more selected from acrylic polymer, methacrylic polymer (e.g., polymethyl methacrylate), acrylonitrile butadiene styrene, polyester, polyamide, polytetrafluoroethylene, polyacetal, cellulose acetate butyrate, polyethylene (e.g., LDPE, HDPE, UHDPE, PEX, etc.), polycarbonate, polyethylene terephthalate, polyethylene terephthalate glycol, polypropylene, polystyrene, or polyvinyl chloride.

In another example, a method of rejuvenating a skin surface can include applying a machine-actuated abrasive matrix on a skin surface, wherein the abrasive matrix comprises abrasive crystals having an average particle size from 25 μm to 250 μm and a plurality of metal-containing particles having an average particle size from 5 nm to 2 μm associated with surfaces of the abrasive crystals. The method can further include the step of depositing a plurality of the metal-containing particles on the skin surface while the machine-actuated abrasive matrix is in contact with the skin surface. The abrasive crystals can include aluminum oxide particles, magnesium oxide particles, sodium chloride particles, sodium bicarbonate particles, or diamond particles and/or can have an average particle size from 40 μm to 180 μm. The abrasive matrix can have a grit count of 80 to 280, for example. The metal-containing particles can include gold particles or gold alloy particles, silver particles or silver alloy particles, copper particles or copper alloy particles, zinc particles or zinc alloy particles, platinum particles or platinum alloy particles, or a combination thereof. Alternatively, the metal-containing particles can be a metal salt of gold, silver, copper, zinc, and/or platinum.

In another example, a method of therapeutically treating a skin surface can include abrading a skin surface with an abrasive matrix of an abrasive skin care appliance. The abrasive matrix in this example includes abrasive crystals having an average particle size from 25 μm to about 250 μm and a plurality of metal-containing particles having an average particle size from 5 nm to 2 μm associated with surfaces of the abrasive crystals. In some examples, abrading the skin surface includes contacting the abrasive matrix with the skin surface while the abrasive matrix is rotating at from 2,000 RPM to 7,000 RPM, from 3,000 RPM to 6,000 RPM, or from 3,400 RPM to 5,010 RPM. In another example, abrading the skin surface includes contacting the abrasive matrix with the skin surface while the abrasive matrix is vibrationally oscillating at from 2,000 VPM to 12,000 VPM, or from 3,000 VPM to 6,000 VPM. In another example, abrading the skin surface includes manually abrading the skin surface by contacting the abrasive matrix with the skin surface without the use of electrical power.

It is noted that when discussing the various abrasive skin care appliances herein, and any kits, systems, methods, etc., related thereto, these various more specific discussions can be considered applicable to one another whether or not they are explicitly discussed in the context of that example. Thus, for example, when discussing the metal-containing particles in the context of one example, such disclosure is also relevant to and directly supported (but not limiting) in the context of other examples, and vice versa. It is also understood that terms used herein will take on their ordinary meaning in the relevant technical field unless specified otherwise. In some instances, there are terms defined more specifically throughout the specification or included at the end of the present specification, and thus, these terms have a meaning as described herein.

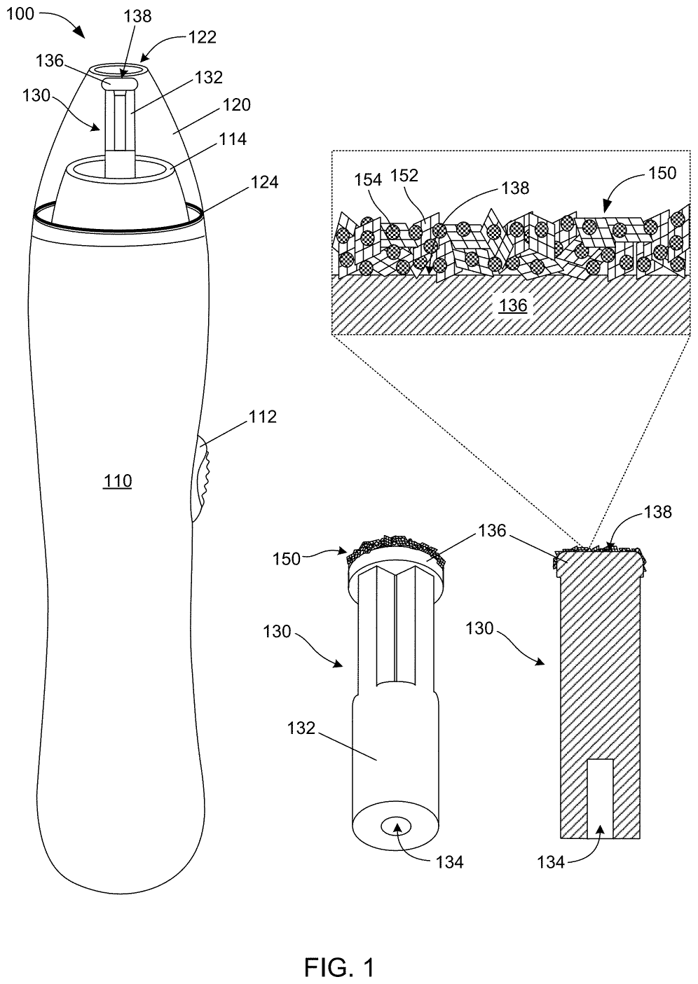

Turning now to one example, as shown in FIG. 1, a microdermabrasion device 100 is shown that includes an abrasive insert 130. In one example, the microdermabrasion device with an attached abrasive insert can be the abrasive skin care appliance as described herein, and in other modular examples, the abrasive insert can be the abrasive skin care appliance. In further detail regarding the microdermabrasion device, this device can include, for example, a microdermabrasion controller 110, which in this example is in the form of a handle, including a switch 112. The switch can be multi-modal, meaning it can control revolutions per minute (RPMs) of the abrasive insert within a range or RPM values, or at a predetermined or fixed RPM, etc. The switch can further control suction intensity, for example. The switch can operate control circuitry, such as a printed circuit board, and the device as a whole can be powered by either direct current (e.g., a rechargeable battery or other battery pack), or by alternating current (e.g., 110 V, 220 V, etc.). The motor and/or vacuum pumps that control abrasion insert RPMs and suction are not shown as they are contained within an outer shell of the microdermabrasion controller. Example revolutions per minute (RPM) that can be used can be from 2,000 RPM to 7,000 RPM, 3,000 RPM to 6,000 RPM, or from 3,400 RPM to 5,010 RPM, for example. As mentioned, the microdermabrasion device can further include a suction head 114, which can include an opening sized to provide a desired suction profile. The opening can also be used to receive the abrasive insert and connect it to a rotational motor (not shown). Also not shown, the vacuum pump and suction head can also be associated with a replaceable filter to prevent skin debris from clogging the suction mechanism (e.g., the filter can be replaced or washed/rinsed for further use). Suction can be applied at any level that is suitable for removing skin debris from the skin surface during the microdermabrasion process.

The microdermabrasion device 100 in this example can also include a suction cap 120 (shown as a clear suction cap in this example) that is fitted, threaded, or otherwise joined with the microdermabrasion controller 110 and in some examples, sealed by an O-ring 124. The cap can include a suction nozzle that is sized to be slightly larger or otherwise similarly sized relative to a disc head facial surface 138 of the abrasive insert 130. The abrasive insert is shown as attached to the microdermabrasion controller through the suction head but could be connected in various other ways to the rotational motor. Further detail related to the abrasive insert is shown in FIG. 1 in lower perspective and cross-sectional views. The abrasive insert can include, for example, an insert body 132 which defines an insert engagement portion 134. The insert engagement portion can be shaped to be received by a rotational rod (not shown) within the microdermabrasion controller, for example. Thus, upon rotating the rotational rod, the abrasive insert spins accordingly. The abrasive insert can also include a disc head substrate 136 that provides the disc head facial surface 138 to which an abrasive matrix 150 can be applied, or as shown in this example, embedded. As an abrasive matrix, the abrasive crystals and the metal-containing particles can be present at a weight ratio from 3:1 to 500:1, from 5:1 to 250:1, from 10:1 to 200:1, from 20:1 to 125:1, for example. The abrasive matrix can also be prepared to have a grit count from 80 to 280, from 80 to 160, from 160 to 280, from 100 to 200, from 120 to 240, or from 120 to 200, for example. As mentioned, the abrasive matrix in these examples include abrasive crystals 152 and metal-containing particles 154.

The abrasive crystals 152 can include, for example, aluminum oxide particles, magnesium oxide particles, sodium chloride particles, sodium bicarbonate particles, diamond particles, and/or the like. The abrasive crystals can have an average particle size from 25 μm to 250 μm, from 30 μm to 200 μm, from 40 μm to 180 μm, from 50 μm to 150 μm, or from 60 μm to 120 μm, for example. In one example, the abrasive crystals can be selected or prepared to be inert relative to the skin surface in that they do not interact with the skin surface chemically, but rather only physically interact with the skin during use as an abrasive. For example, the abrasive crystals can be hypoallergenic, or even considered to be medical grade, ultra-hypoallergenic, and/or ultra-hygienic, in some examples.

The metal-containing particles 154 can include gold particles or gold alloy particles, silver particles or silver alloy particles, copper particles or copper alloy particles, zinc particles or zinc alloy particles, platinum particles or platinum alloy particles, or a combination thereof. Alternatively, the metal-containing particles can be a metal salt of gold, silver, copper, zinc, and/or platinum. The metal-containing particles can have an average particle size from 5 nm to 2 μm, from 5 nm to 500 nm, from 10 nm to 2 μm from, from 10 nm to 1 μm, from 10 nm to 500 nm, from 15 nm to 350 nm, from 10 nm to 100 nm, from 100 nm to 500 nm, or from 200 nm to 1 μm, for example. In one example, the metal-containing particles can be elemental gold or an elemental gold alloy of at least 50 wt % gold and may have an average particle size from 5 nm to 150 nm. In another example, the metal-containing particles can be elemental silver or an elemental silver alloy of at least 50 wt % silver and may have an average particle size from 50 nm to 250 nm. In another example, the metal-containing particles can be elemental copper or an elemental copper alloy of at least 50 wt % copper and may have an average particle size from about 100 nm to about 500 nm. In another example, the metal-containing particles can be elemental platinum or an elemental platinum alloy of at least 50 wt % platinum and may have an average particle size from about 50 nm to about 500 nm. In another example, the metal-containing particles can be zinc oxide, zinc pyrithione, or elemental zinc nanoparticles or zinc alloys of at least 50 wt % zinc and may have an average particle size from about 50 nm to about 500 nm. Thus, the metal-containing particles can be elemental metal, elemental metal alloys, or can be a salt of a metal as described above. As an alloy, the metals selected for use can include one of gold, silver, copper, zinc, or platinum; or the metals selected for use can be two or more of gold, silver, copper, zinc, or platinum. Thus, there can be other metals present in the alloys, and in some instances, even non-metals, such as semi-metals or dopants (e.g., silicon, oxygen, etc.). Furthermore, the metal-containing particles can likewise be in the form of a salt, as mentioned.

In further detail regarding the metal-containing particles 154, they may be used for their antimicrobial effect to the surface of the abrasive matrix 150, but these metals can additionally or alternatively provide a therapeutic benefit to the skin surface as well. For example, gold is an antioxidant and an antimicrobial, and can assist with the reduction of acne and in some instances, allergies. Gold can also stimulate skin cells and increase elasticity in the skin. Thus, when used in conjunction with microdermabrasion, by contacting the skin with gold particles and further, by leaving some residual gold particles on the skin, cellular stimulation and skin elasticity can be promoted as the abraded skin regenerates. Likewise, silver particles can benefit the skin due to its antimicrobial properties, which can be beneficial in preventing blackheads, acne, and the like at the abraded skin surface. Copper, on the other hand, unlike silver that is typically antimicrobial via ion diffusion, is a good contact killer of microbes, such as bacteria, viruses, fungus, etc. Furthermore, copper helps develop collagen and elastin, assists with repair of skin, and can promote the production of hyaluronic acid. Zinc can help generally with acne, heal cuts and wounds, treat topical irritations, itches, etc., and can be used to treat injuries, skin sores, rashes, seborrheic dermatitis, psoriasis, eczema, dandruff, etc. Zinc can also promote skin regeneration and provide a protective coating from the sun. Platinum can also be used for its antioxidant activity, reducing inflammation and free-radical damage that may otherwise occur at the outermost layer of the skin after treatment. Platinum can also promote healthier or stronger skin having a brighter appearance.

Furthermore, the abrasive matrix can likewise be formulated to include other metals or metal salts in addition to the metal-containing particles 154 described above (e.g., gold, silver, copper, zinc, platinum, alloys thereof, salts thereof, etc.) that may or may not be therapeutically beneficial or antimicrobial. For example, the abrasive matrix can be formulated to include from 0.01 wt % to 20 wt % of zinc, iron, calcium, magnesium, manganese, chromium, potassium, sodium, or the like. Likewise, the abrasive matrix can include alloys of metals with oxygen, silicon, sulfur, phosphorus, etc. Other antioxidant compounds can likely be included in relatively small concentrations as well (e.g., 0.01 wt % to 5 wt %).

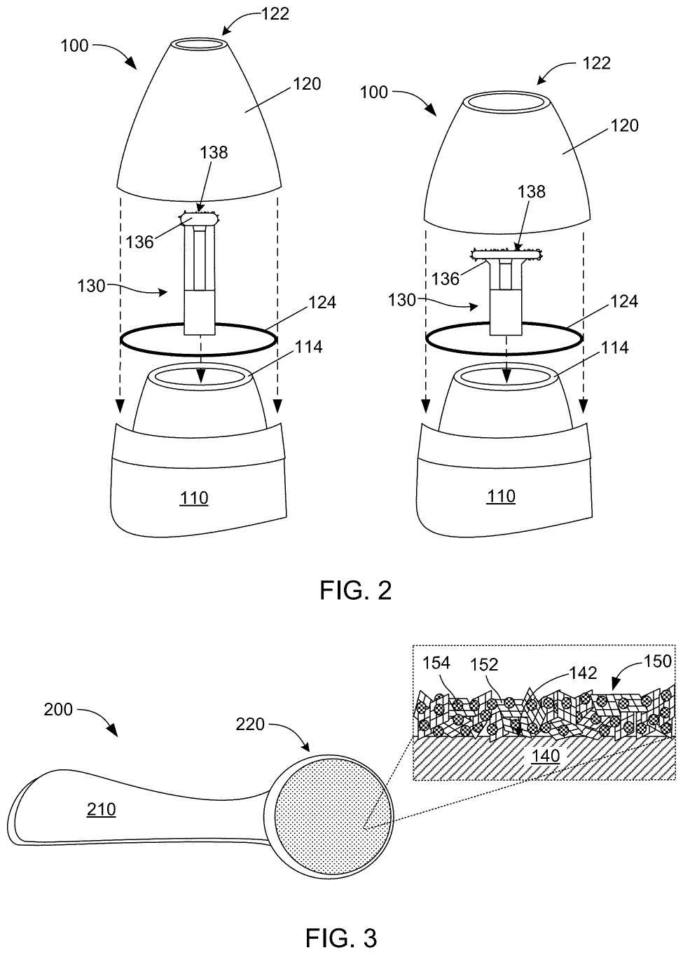

In further detail, the abrasive matrix 150 can be positioned on and affixed to the disc head facial surface 138 (or pad facial surface 142 as shown in the FIGS. 3-5 examples) by any of a number of attachment mechanisms, depending to some degree on the substrate material selected and configured to support the adhesive matrix. Example materials that can be used for the disc head substrate 136 shown in FIGS. 1-2, or pad substrate 140 shown in FIGS. 3-5, or other substrate configuration, include any of a number of materials such as plastic, metal, glass, stone, fabric, composites thereof, or the like. With these or other materials, the abrasive matrix can be applied using an adhesive, applied by embedding the abrasive matrix in the substrate material, applied by fastening a coupon of the abrasive matrix to the substrate, or by any other methodology.

In one specific example, the disc head substrate 136 of FIGS. 1-2, or the pad substrate 140 of FIGS. 3-5, or other substrate configuration, can be a polymeric (e.g., plastic), substrate material that is thermally or chemically softenable. Thus, the abrasive matrix 150 can be embedded in the plastic substrate material by a thermal process, where the substrate is warmed to a softening temperature so that the aluminium oxide particles can be pressed or otherwise sunk into the softened plastic substrate at the disc head facial surface 138 (FIGS. 1-2) or the respective pad facial surface 142 (FIGS. 3-5). As an alternative method, the disc head facial surface or the pad facial surface of the respective polymeric substrate can be chemically softened and dipped into a powder bed of the abrasive matrix particles (abrasive crystals 152 and metal-containing particles 154) with some pressure, and then allowed to dry, leaving the abrasive matrix embedded therein. Application of the chemical or solvent used to soften the polymeric substrate and pressing the plastic substrate into the powder bed of the abrasive matrix particles can be done sequentially or simultaneously, for example. If done sequentially, then chemical softening solvent can be applied to the substrate surface first, followed by pressing the softened plastic surface into the power bed of abrasive matrix particles. If done simultaneously, then a mixture of the chemical softening solvent and the abrasive matrix particles can be used, provided there is enough chemical softening solvent present to sufficiently soften the substrate surface for embedding the particles into the surface. Contact between the substrate surface and the chemical softening solvent can be for a relatively short period of time, such as from 10 seconds to 2 minutes, 10 seconds to 50 seconds, etc., and then the substrate can be removed for hardening.

Thus, the facial surface 138 or 142 of the respective substrate 136 or 140 can include a softenable plastic material, such as a solvent-softenable plastic material and/or a thermal-softenable plastic material. In some instances, the same plastic material can be thermally and/or solvent-softenable, and thus, the process may include both solvent softening and thermal softening in combination. Example materials that can be selected for use include acrylic polymer, methacrylic polymer (e.g., polymethyl methacrylate), acrylonitrile butadiene styrene, polyacetal, cellulose acetate butyrate, polyethylene (e.g., LDPE, HDPE, UHDPE, PEX, etc.), polycarbonate, polyethylene terephthalate, polyethylene terephthalate glycol, polypropylene, polystyrene, polyvinyl chloride, or combinations thereof. These materials can all be chemically softened and/or thermally softened. Materials such as polyamides (or nylons) or polytetrafluoroethylene (e.g., Teflon) may be more suitable for thermal softening than chemical softening, but to the extent softenable with solvent and/or heat, any plastic or polymeric material can be used.

For thermal softening examples, this can be carried out by bringing the temperature of the facial surface of the substrate to a temperature where the polymeric material is softened, but typically is not fully melted. In some examples, the point at which the substrate material is softened can be defined by its softening temperature, sometimes referred to as the Vicat softening temperature. The Vicat softening temperature can be determined, for example, by determining the temperature at which a flat-ended needle with a 1 mm² circular cross-section can penetrate 1 mm of the polymer sample under a load of 10 Newtons for the Vicat A test or a load of 50 Newtons for the Vicat B test. Likewise, standards that can be used include ASTM D 1525 and ISO 306, which are essentially an equivalent test. To the extent multiple methodologies provide different results, an average can be used to provide the softening temperature for a given plastic material.

On the other hand, if softening the facial surface of the polymeric substrate with a solvent (or chemical), example solvents that can be selected for use include 1,2-dichloroethane, acetone, cyclohexanone, dichloromethane, methyl ethyl ketone (MEK), methyl benzene, tetrahydrofuran, or a combination thereof. The solvent selected can be matched with the plastic material selected and can be applied at a concentration and for a time period to soften the substrate sufficiently to embed the abrasive matrix therein.

Turning now more specifically to FIG. 2, two different top portions of the microdermabrasion device 100 are shown. The structures shown in these two alternative examples have the same features shown in FIG. 1, but this FIG. shows that the disc head substrate 136 and associated disc head facial surface 138 can be of multiple sizes or configurations, and furthermore, that the suction cap 120 and/or suction nozzle 122 can be alternatively sized, shaped, positioned, etc., to accommodate the size, location, configuration, etc., of the disc head facial surface.

Referring now to FIG. 3, a manually actuatable microdermabrasion device 200 is shown, which in this instance is in the form of an abrasive brush with a handle portion 210 and a head portion 220. The handle portion can be gripped by a user to manually actuate (circular motion, side to side motion, or otherwise) the head portion. The head portion can include a pad substrate 140 with an abrasive matrix 150 associated with a pad facial surface 142 thereof. Again, the abrasive matrix can include abrasive crystals and metal-containing particles, as previously described. The head portion, or more specifically, the pad substrate, can be of any size (e.g., facial surface area) or shape (e.g., circular, square, oval, geometric shape, elongated shape, etc.) suitable for a given microdermabrasion application (e.g., from 1 cm² to 50 cm², from 4 cm² to 25 cm², from 10 cm² to 50 cm², or from 2 cm² to 10 cm²). In some examples, there may be larger or smaller skin-contact surface areas.

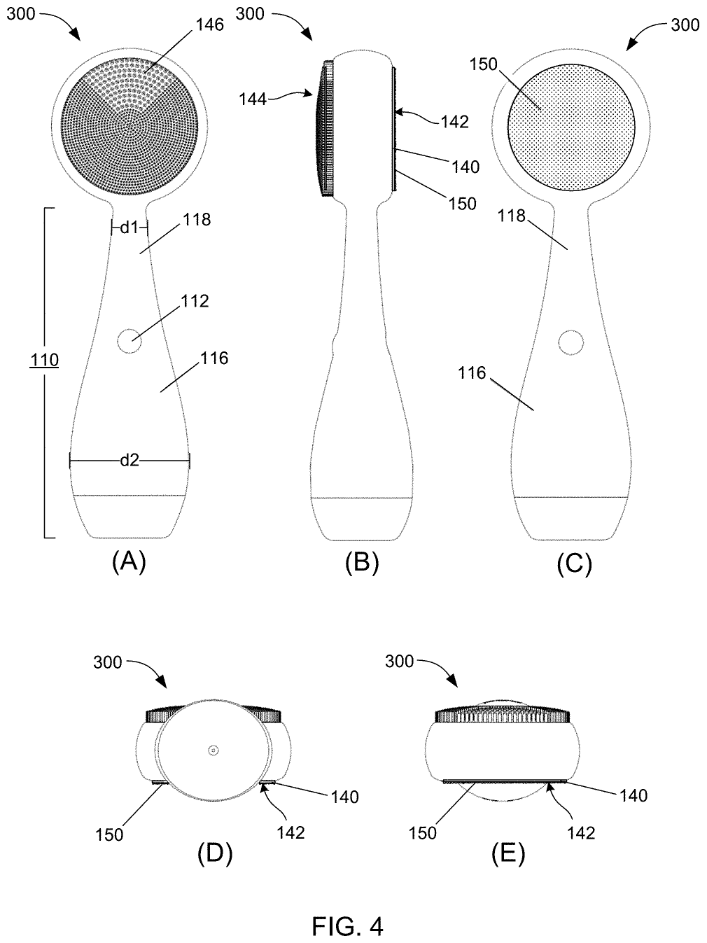

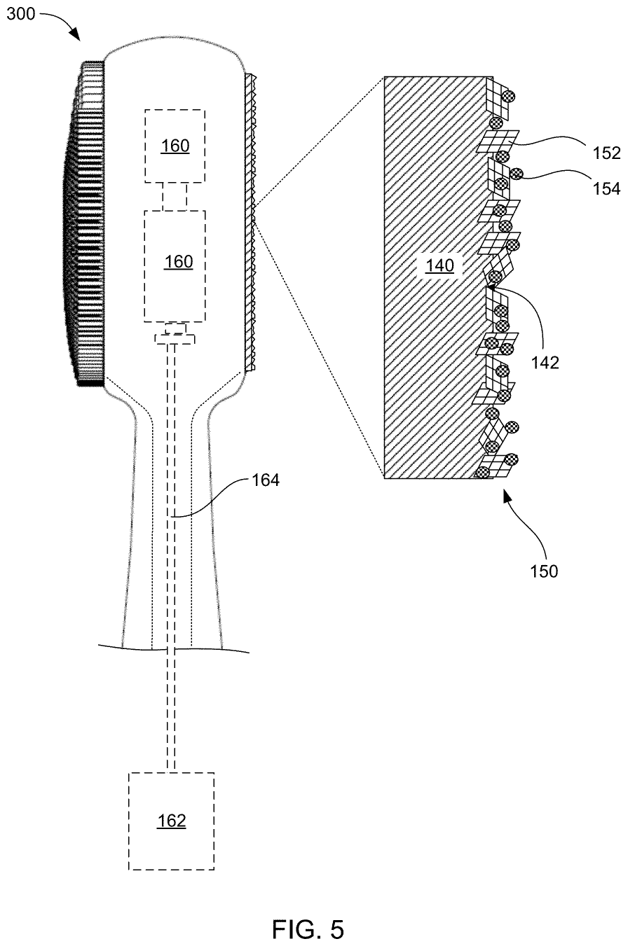

Turning now to FIGS. 4 and 5, five (5) different views of a microdermabrasion device 300 are shown in FIG. 4 (e.g., front view (A), side view (B), back view (C), bottom view (D), and top view (E)), as well as a more detailed view in FIG. 5. In this example, the microdermabrasion device includes multiple skin care surfaces, including the microdermabrasion surface on one side and a skin cleansing surface on the other side. This is provided by example only, as the device could be a dedicated microdermabrasion device, or alternatively, could include two surfaces that are both for microdermabrasion (each side of a different size, shape, and/or abrasive grit count, for example). With this in mind, the microdermabrasion device shown in FIG. 4 includes a microdermabrasion controller 110, which in this example is in the form of a handle which includes a switch 112, which can be a multifunctional switch (e.g., on/off, variable oscillation, variable temperature, etc.). The switch can operate control circuitry (not shown, but shown at 162 in FIG. 5), such as a printed circuit board, and the device as a whole can be powered by either direct current (e.g., a rechargeable battery or other battery pack), or by alternating current (e.g., 110 V, 220 V, etc.). The handle portion in this example includes a neck portion 118 and a grip portion 116. As mentioned, in this example, the handle supports a head that includes both a microdermabrasion appliance and a skin cleansing appliance. The skin cleansing appliance can include, for example, an array of skin-cleansing bristles 146. The microdermabrasion surface can include a microdermabrasion portion including a pad substrate 140 having a pad facial surface 142 with an abrasive matrix 150 associated therewith. As can be seen in more detail in FIG. 5, the abrasive matrix can include abrasive crystals 152 and metal-containing particles 154, as previously described.

In some examples, the microdermabrasion device can include a vibrational motor 160 (not shown in FIG. 4 but shown in FIG. 5) to provide vibratory oscillation to a head of the microdermabrasion device. The motor can be within the head, adjacent to the pad substrate 140, for example. The motor can be supported, in one example, by a support rod 164, for example. The support rod can be flexible, but rigid enough to provide support between the grasping portion of the handle and the head. The support rod in certain more specific examples can be a solid metal rod, a tube-like rod, or a spring-like rod. The support rod can have any cross-sectional shape, such as a circular cross-section, a square cross-section, a rectangular cross-section, a triangular cross-section, a pentagonal cross-section, a hexagonal cross-section, and so forth. The support rod can be metal, polymer (e.g., plastic, rubber, etc.), ceramic, etc. As mentioned, the cross-sectional configuration can be solid, tubular, or a combination of both. The support rod and/or vibrational motor can be mechanically fastened to the head at the head support shell, for example, and the support rod can be mechanically fastened to a location within the grip portion.

In this particular example with respect to the microdermabrasion controller 110 (handle portion), note that the neck portion 18 includes a location that is narrower (shown at d1) than at a location of the grip portion 16 (shown at d2). The diameter (converted to radius; r=d/2) in this example can be used to calculate the cross-sectional areas of the two respective locations. It is noted that if the cross-sectional areas are not defined by a round geometry (perpendicular to the axis of the handle), then other mathematical calculations can be used to determine the respective cross-sectional areas. In this example, as can be seen, the much narrower cross-sectional area (d1) at the neck portion (at least at one location) can be from 4 to 15 times smaller, from 6 to 12 times smaller, or from 8 to 10 times smaller, than a cross-sectional area (d2) of the grip portion (at least at one location). In this specific example, based on the relative diameters shown, the cross-sectional area at (d1) can be calculated to be from about 8 to 10 times smaller than at (d2). In accordance with examples herein, by using a narrowed neck portion in conjunction with a support rod, and having space between the support rod and the material that defines the narrowed neck portion, the vibrational energy at the head of the microdermabrasion device can be dampened so that the handle exhibits less vibrational energy. In some examples, the material used to provide the body or shape to the grip portion and the neck portion can be a rubber or other soft material that can act to assist with vibrational dampening.

Vibrational dampening can be helpful with this type of device, as the presence of particularly the secondary skin care appliance and associated structures that may be present to operate this appliance can add enough weight to the head so as to make vibrational transference more noticeable to users. Furthermore, this can be particularly the case when the vibrational motor is potentially delivering a vibrational oscillation frequency from 2,000 vibrations per minute (VPM) to 12,000 VPM. With that stated, example VPM frequencies can be implemented at a fixed frequency, or with multiple fixed frequency settings (e.g., 2 discrete settings, 3 discrete settings, 4 discrete settings, etc.), or with variable frequency settings (e.g., frequencies dialed up and down), as designed. Example frequency ranges can be from 2,000 VPM to 12,000 VPM, from 4,000 VPM to 12,000 VPM, from 6,000 VPM to 12,000 VPM, from 7,000 VPM to 12,000 VPM, from 2,000 VPM to 10,000 VPM, from 4,000 VPM to 10,000 VPM, from 6,000 VPM to 10,000 VPM, from 7,000 VPM to 10,000 VPM, from 2,000 VPM to 8,000 VPM, from 4,000 VPM to 8,000 VPM, from 6,000 VPM to 8,000 VPM, from 7,000 VPM to 8,000 VPM, from 2,000 VPM to 6,000 VPM, from 3,000 VPM to 6,000 VPM, from 4,000 VPM to 6,000 VPM, from 6,000 VPM to 10,000 VPM, from 7,000 VPM to 9,000 VPM, from 7,000 VPM to 8,000 VPM, or from 8,000 VPM to 12,000 VPM.

If there are multiple types of skin care appliances, as shown by example in FIGS. 4 and 5, the secondary skin care appliance can be a skin-cleansing appliance, a heating appliance, a cooling appliance, a massaging appliance (vibratory or unpowered), electromagnetic energy appliance, an ionic infusion appliance, a phototherapy appliance, a porous scrubbing appliance, or a combination thereof. The secondary skin care appliance can likewise be another microdermabrasion appliance of a different size, shape, grit level, abrasive matrix composition, etc. For example, one side may include gold particles and the other side may include copper particles, or one side may include silver particles and the other side may include non-metal-containing particle, or one side may include gold particles and the other side may be a different type of microdermabrasion appliance altogether (e.g., etched metal surface). In further detail, there can be multiple skin care appliances on the rotational microdermabrasion device shown in FIGS. 1 and 2, as well as the manually actuatable microdermabrasion device shown in FIG. 3, but this feature is shown by example only in FIGS. 4 and 5 so as to not obscure the description related to the microdermabrasion appliances described herein, but it is noted that any of the microdermabrasion devices shown and described herein or otherwise configured can include multiple skin care appliances. Furthermore, a microdermabrasion device may include a microdermabrasion appliance that is operated with a motor (rotation or vibration) and a second microdermabrasion appliance may be manually operated, or a microdermabrasion device may include one rotational appliance and one vibratory appliance, etc.

Regarding skin-cleansing appliance examples, there can be (as the secondary skin care appliance) an appliance with skin-cleansing bristles 146, as shown in FIGS. 4 and 5 by example. These bristles can have a length of from 1/16 inch to ⅜ inch, from ⅛ inch to ¼ inch, from 1/16 inch to ¼ inch, or from ⅛ inch to ⅜ inch, for example. The bristles can have a uniform density at the first external region, or can be arranged in multiple sub-regions within the first external region with corresponding different bristle densities, as shown in FIG. 4 (with a top fan-shaped region having a lower bristle density than the rest of the circular shaped “first external region”). An average bristle density at the first external region can be from 100 to 600, from 150 to 500, from 200 to 400 or from 250 to 400 bristles per square inch, regardless if there are multiple sub-regions with different densities. In further detail, however, if the first external region of bristles includes two discrete sub-regions of bristles, then the first sub-region can have a bristle density from 150 to 700, from 200 to 600, from 200 to 500, or from 250 to 400 bristles per square inch, and furthermore, the second sub-region can have a bristle density from 40 to 300, from 50 to 250, from 75 to 250, or from 100 to 200 bristles per square inch, for example. Typically, the second sub-region in this example will have a lower bristle density than the first sub-region. The skin-cleansing bristles at the first external region can have a skin-contact surface area of from 2 square inches to 35 square inches, from 3 square inches to 30 square inches, from 3 square inches to 25 square inches, from 3.5 square inches to 20 square inches, from 3.5 square inches to 10 square inches, from 5 square inches to 35 square inches, or from 10 square inches to 35 square inches, for example.

In further detail, the skin-cleansing bristles can be constructed of a rubber material, such as a polysiloxane or a silicone rubber. For example, the silicone rubber can be hypoallergenic (e.g., medical grade, ultra-hypoallergenic, ultra-hygienic, and/or odor-resistant silicone rubber). Silicone rubbers are an elastomer of silicone, carbon, hydrogen, and oxygen. Silicone rubbers can include fillers to modulate material properties and/or cost. If fillers are added, they can be added keeping in mind the use as a skin-cleansing brush, and can be selected to be hypoallergenic, hygienic, etc. With this particular type of brush (e.g., a polysiloxanes or silicone rubber bush), the brush can be constructed so that it does not need to be replaced. That stated, it can also be constructed to be modular for periodic replacement, if desired. With these types of silicone rubbers, it can be gentle enough to use on nearly all types of skin, particularly healthy, unbroken skin. In some instances, it can even be gentle enough to use on damaged or diseased skin. The skin-cleansing bristles can be constructed of either the same material or a different material than used as a sleeve over the handle and/or portions of the head. Though silicone rubber can be used effectively, it is noted that other types of bristle brushes can be used as well, depending on the desired skin application The skin-contact surface area of the skin-cleansing brushes can be, for example, from 1 square inch to 35 square inches, from 2 square inches to 30 square inches, from 5 square inches to 25 square inches, or from 10 square inches to 35 square inches, for example.

It is noted that the microdermabrasion device 300 shown in FIGS. 4 and 5 include skin cleansing bristles on an opposite surface relative to the abrasive matrix 150. However, as mentioned, the second side need not be functional, or it can be another microdermabrasion appliance, or it can include another alternative secondary skin care appliance. Examples of other secondary skin care appliances may include a skin treatment appliance, which may be a heat treatment appliance, a cooling treatment appliance, a massaging appliance (e.g., used without heat or cooling). If using a heat treatment appliance, heating can be carried out, for example, using a heat treatment appliance that contacts the skin surface at a temperature from 36° C. to 44° C. or from 38° C. to 42° C. If the heat treatment appliance is a good heat conductor, like metal, then the temperature of the heat conductor can be about the same as the target temperature for skin contact by the heating pad. If the heat treatment appliance is not a good conductor of heat, such as a semi-precious stone, then the temperature at the heat conductor can be higher so that the surface of the semi-precious stone reaches the target skin therapeutic temperature (e.g., from 36° C. to 44° C. or from 38° C. to 42° C.). Cooling treatment appliance temperatures may be used at temperatures from −10° C. to 20° C., from −5° C. to 15° C., or from 0° C. to 15° C., or from 0° C. to 10° C., taking user safety into account, as the mean temperature where frostnip can occur with prolonged exposure may be at about −10° C. for many subjects. A temperature sensor can be present to monitor and/or help control the temperature of the heating and/or cooling pad, for example. If used, therapeutic pads may provide a therapeutic effect relative to the skin via temperature, via application of serums or other compounds to the skin, and/or by contact of the material itself to the skin. Some materials, temperatures, and or application compounds, for example, may be therapeutic when used before and/or after microdermabrasion treatment. Example skin treatment pads can be of any material, such as rubber (e.g., silicone rubber), metal (e.g., gold, silver, copper, zinc, platinum, alloys thereof, salts thereof, etc.), therapeutic semiprecious stones (e.g., quartz, such as rose quartz, or jade), porous stones (e.g., pumice stone), plastic, electroplated plastic, glass, ceramic, soft natural material (e.g., loofah, sponge, etc.), soft synthetic material, etc.

Other secondary skin care appliances may likewise be used (instead of or in addition to the skin-cleansing bristles shown in FIGS. 4 and 5). Such appliances may include electromagnetic energy appliances (e.g., phototherapy and/or radio frequency), ionic infusion appliances (e.g., microcurrent, a massaging appliance, etc.), LED phototherapy appliances (e.g., red light, blue light, etc.), soft porous scrubbers (e.g., loofahs, sponges, poufs, meshes, etc.), anti-microbial soft porous scrubbers, massaging appliances with massaging relief features, or the like.

In accordance with examples of the present disclose, the devices herein can be used as part of a system or kit, where the skin care device is co-packaged or used together with any of a number of skin care fluids, such as serums, essences, ampoules, moisturizers, creams or ointments, topical pharmaceutics, topical nutricosmetics or cosmeceutics, or the like. These types of skin care fluids can carry compounds to the skin, and can include a liquid carrier (e.g., aqueous carrier or oil-based carrier), which carry an active ingredient to (or into) the epidermis. Example oils can include argan oil, rose oil, vitamin oil, seed oil (e.g., pomegranate seed oil, rose hip see oil, etc.), jojoba oil, nut oil (e.g., macadamia nut oil, kukui nut oil, etc.), oil from fruit or flowers (e.g., apricot kernal oil, orange oil, lemon oil, neroli flower oil, jasmine oil, coconut oil, etc.), aloe, hemp oil, or the like. The oil can act as the active ingredient in part or can be merely a carrier for an active ingredient. Active ingredients, on the other hand, can include metal(s) and other minerals, antioxidant(s), fatty acids, vitamins (e.g., vitamin E, vitamin C, vitamin A, etc.), drugs, other anti-aging compounds, anti-inflammatory agents (e.g., aloe, green tea, etc.), organic acids, e.g., mandelic acid, malic acid, hyaluronic acid, salicylic acid, etc., skin-brightening compounds, anti-acne compounds (e.g., salicylic acid), antimicrobial compounds (e.g., colloidal or ionic silver compounds), hydrating compounds, proteins, peptides, amino acids, amino acid chelates or other chelates, or the like. In one example, the skin care devices described herein can include an anti-aging or an anti-wrinkle serum, such as a serum with mandelic acid and/or malic acid. In another example, the serum can include a peptide.

In accordance with another example, a method of making an abrasive skin care appliance can include combining abrasive crystals with metal-containing particles at a weight ratio from 3:1 to 500:1, or from 5:1 to 250:1 to form an abrasive particulate blend, wherein the abrasive crystals have an average particle size from 25 μm to 250 μm, or from 30 μm to 200 μm, and the metal-containing particles have an average particle size from 5 nm to 5 μm. The method can also include softening a facial surface of an appliance substrate and pressing the facial surface into the abrasive particulate blend while the facial surface is in a softened state to form an abrasive matrix partially embedded in the facial surface.

In another example, a method of therapeutically treating a skin surface can include abrading a skin surface with an abrasive matrix of an abrasive skin care appliance. The abrasive matrix in this example includes abrasive crystals having an average particle size from 25 μm to 250 μm, or from 30 μm to 200 μm, and a plurality of metal-containing particles having an average particle size from 5 nm to 5 μm associated with surfaces of the abrasive crystals. In certain examples, abrading the skin surface includes contacting the abrasive matrix with the skin surface while the abrasive matrix is rotating at from 2,000 RPM to 7,000 RMP, from 3,000 RPM to 6,000 RPM, or from 3,400 RPM to 5,010 RPM. In another example, abrading the skin surface includes contacting the abrasive matrix with the skin surface while the abrasive matrix is vibrationally oscillating at from 2,000 VPM to 12,000 VPM, or in one example, from 3,000 VPM to 6,000 VPM. In another example, abrading the skin surface includes manually abrading the skin surface by contacting the abrasive matrix with the skin surface without the use of electrical power.

Notably, the details described herein related to the microdermabrasion device and related appliances can be used or prepared in conjunction with these and other methods.

It is noted that, as used in this specification and the appended claims, the singular forms “a,” “an,” and “the” include plural referents unless the context clearly dictates otherwise. In describing and claiming the teachings of the present disclosure, the following terminology will be used in accordance with the definitions set forth below.

Dimension, amounts, concentrations, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited.

As an illustration, a numerical range of “about 10 to about 50” should be interpreted to include not only the explicitly recited values of about 10 to about 50, but also to include individual values and sub-ranges within the indicated range. Thus, included in this numerical range are individual values such as 20, 30, and 40 and sub-ranges such as from 10-30, from 20-40, and from 30-50, etc. This same principle applies to ranges reciting only one numerical value. Furthermore, such an interpretation should apply regardless of the breadth of the range or the characteristics being described.

### EXAMPLE

### Example 1

### Preparation of Microdermabrasion Discs with Gold Nanoparticles

Two different microdermabrasion inserts with disc-shaped facial surfaces were used in this example which were configured similar to that shown in FIG. 2, one having a 6 mm diameter facial surface and one having a 12 mm diameter facial surface. The microdermabrasion inserts, and particularly the facial surfaces thereof, were constructed from a solvent-softenable plastic. Both facial surfaces were treated with aluminum oxide crystals and gold particles. The aluminum oxide crystals and the gold particles individually had a surface morphology as shown in FIG. 2, and the abrasive matrix had a particlized face at about 120 grit.

In this example, the two facial surfaces were dipped in solvent for 20 seconds to soften the facial surfaces, which were then wet pressed into a homogenous blend of the aluminum oxide crystals and the gold particles at a weight ratio of about 40:1. Notably, a thermally-softenable plastic, such as a thermoplastic, can likewise be used to soften the plastic with heat, but in this example, the plastic used was selected for solvent-softenable softening properties. After removing the facial surfaces from the homogenous blend of aluminum oxide crystals and gold particles, the microdermabrasion discs were then allowed to dry overnight to form the microdermabrasion discs which included both the aluminum oxide crystals and the gold particles. The homogenous blend of the aluminum oxide crystals and gold particles on both microdermabrasion discs were visually sampled under magnification and confirmed to have been applied at weight ratios of about 49:1. Thus, a slightly higher ratio of aluminum oxide crystals to gold particles ended up in the abrasive matrix applied to the facial surface of the appliance. This may be due to the fact that the larger aluminum oxide crystals become embedded into the facial surface of the appliance, whereas the gold particles tend to become part of the matrix as being primarily applied to surfaces of the aluminum oxide crystals.

### Example 2

### Retention of Gold Nanoparticles in Microdermabrasion Discs

Using X-ray Photoelectron Spectroscopy (XPS) imaging at 400× power, imagery was collected from portions of the large microdermabrasion disc particlized face and portions of the small microdermabrasion disc particlized face. The first few (outermost) micrometers of depth was captured in the imagery. The two microdermabrasion discs were then each used 5 times on a subject's facial skin according to manufacturer instructions, and the two microdermabrasion discs were then similarly reimaged using XPS imaging at 400× power. The parts by weight of the aluminum oxide crystals and the gold particles are provided in Tables 1A and 1B, as follows:

TABLE 1A

| | Before Use | After Use | |
|---|---|---|---|
| | (parts by | (parts by | % Change |
| Large Disc | weight) | weight) | (by weight) |
| Aluminum Oxide Crystals | 100 | 88 | −12% |
| Gold Nanoparticles | 2 | 1.7 | −15% |

TABLE 1B

| | Before Use | After Use | |
|---|---|---|---|
| | (parts by | (parts by | % Change |
| Small Disc | weight) | weight) | (by weight) |
| Aluminum Oxide Crystals | 108 | 96 | −11% |
| Gold Nanoparticles | 2.1 | 1.8 | −14% |

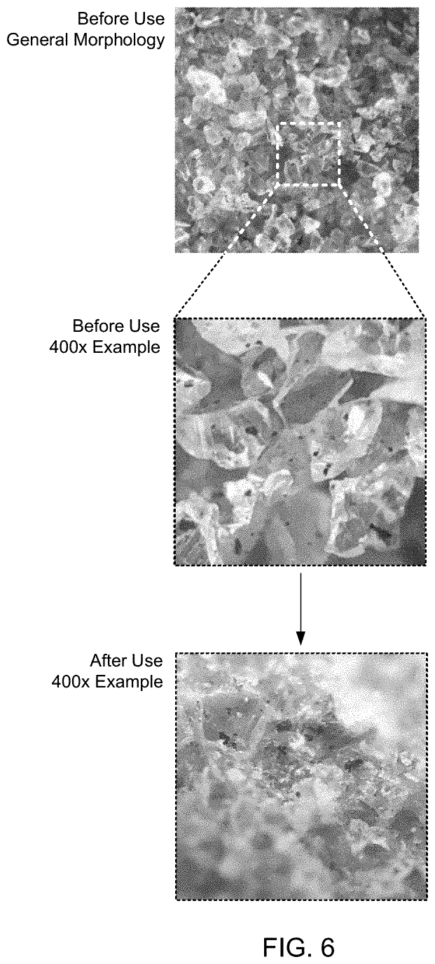

FIG. 6 shows XPS images before use (one at 400× power and one at a lower power level), as well as one 400× XPS image after use. It is noted that these images are not necessarily of the same quadrants of a common disc or even from the same disc before and after use, but rather were selected merely to show clear quadrants from various discs and/or quadrants that could be used to collect the actual data shown in Tables 1A and 1B.

As evident from the data in Tables 1A and 1B, and in connection with imagery approximated by that shown by example in FIG. 2, after use, both large and small discs showed essentially proportional loss of aluminum oxide crystals and gold particles. As can be seen also in the “after use” image of FIG. 6, residue of skin debris becomes deposited on the surface of the disc, obscuring some of the crystals and gold particles. However, the microscope imagery does show that to some extent, gold particles can assemble in clumps or groups on the surface of the aluminum oxide crystals when applied. Furthermore, gold particles are also visible both on the outermost aluminum oxide crystals as well as underlying crystals visible between the outermost aluminum oxide crystals. For comparison, even after use, the gold nanoparticle clusters on the aluminum oxide crystals remain visible, indicating that for the most part, they remained intact during 5 uses. Thus, the overall conclusion may be that as a subject uses these microdermabrasion discs over time, and as aluminum oxide crystals become removed from the facial surface, new layers of aluminum oxide crystals and gold particles become exposed, thus allowing the gold particles to continue to provide continued effectiveness.

It is to be understood that the above-referenced arrangements and examples are only illustrative of the application of the principles of the present disclosure. Numerous modifications and alternative arrangements can be devised without departing from the present disclosure. While the present disclosure has been shown in the drawings and fully described above with particularity and detail in connection with what is presently deemed to be the most practical embodiment(s) of the disclosure, it will be apparent to those of ordinary skill in the art that numerous modifications can be made without departing from the principles and concepts of the disclosure as set forth herein.

What is claimed is:

1. An abrasive skin care appliance, comprising a facial surface having an abrasive matrix attached thereto, wherein the abrasive matrix comprises abrasive crystals having an average particle size from about 25 μm to about 250 μm and a plurality of metal-containing particles having an average particle size from 5 nm to 2 μm associated with surfaces of the abrasive crystals.

2. The abrasive skin care appliance of claim 1, wherein the abrasive crystals include aluminum oxide particles.

3. The abrasive skin care appliance of claim 1, wherein the abrasive crystals include magnesium oxide particles, sodium chloride particles, or sodium bicarbonate particles.

4. The abrasive skin care appliance of claim 1, wherein the abrasive crystals include diamond particles.

5. The abrasive skin care appliance of claim 1, wherein the abrasive crystals have an average particle size from 40 μm to 180 μm.

6. The abrasive skin care appliance of claim 1, wherein the abrasive matrix has a grit count of 80 to 280.

7. The abrasive skin care appliance of claim 1, wherein the metal-containing particles include gold particles or gold alloy particles.

8. The abrasive skin care appliance of claim 1, wherein the metal-containing particles include silver particles or silver alloy particles.

9. The abrasive skin care appliance of claim 1, wherein the metal-containing particles include copper particles or copper alloy particles.

10. The abrasive skin care appliance of claim 1, wherein the metal-containing particles include zinc particles or zinc alloy particles.

11. The abrasive skin care appliance of claim 1, wherein the metal-containing particles include platinum particles or platinum alloy particles.

12. The abrasive skin care appliance of claim 1, wherein the metal-containing particles include salt particles of gold, silver, copper, zinc, or platinum.

13. The abrasive skin care appliance of claim 1, wherein the metal-containing particles have an average particle size 15 nm to 1 μm.

14. The abrasive skin care appliance of claim 1, wherein the abrasive crystals and the metal-containing particles are present in the abrasive matrix at a weight ratio from about 3:1 to about 500:1.

15. The abrasive skin care appliance of claim 1, wherein the abrasive skin care appliance is an insert or a pad shaped for associating with a microdermabrasion device, or wherein the abrasive skin care appliance is attached to a microdermabrasion device.

16. The abrasive skin care appliance of claim 15, wherein the microdermabrasion device is a rotational microdermabrasion device including a rotational motor to rotate the insert or pad and a vacuum to remove skin debris away from the abrasive matrix.

17. The abrasive skin care appliance of claim 16, wherein the rotational motor is rotatable at one or more setting from 2,000 RPM to 7,000 RPM.

18. The abrasive skin care appliance of claim 15, wherein the microdermabrasion device is a vibrational microdermabrasion device a vibrational motor positioned within a head where the abrasive matrix contacts a skin surface.

19. The abrasive skin care appliance of claim 18, wherein the vibrational motor is oscillatable at one or more setting from 2,000 VPM to 12,000 VPM.

20. The abrasive skin care appliance of claim 1, wherein the microdermabrasion device includes a secondary appliance associated therewith.

21. The abrasive skin care appliance of claim 1, wherein the secondary appliance is skin-cleansing appliance, a heat treatment appliance, a cooling treatment appliance, a massaging appliance, electromagnetic energy appliance, an ionic infusion appliance, a phototherapy appliance, a porous scrubbing appliance, or a combination thereof.

22. A method of making an abrasive skin care appliance, comprising:
combining abrasive crystals with metal-containing particles at a weight ratio from 3:1 to about 500:1 to form an abrasive particulate blend, wherein the abrasive crystals have an average particle size from 25 μm to about 250 μm and the metal-containing particles have an average particle size from 5 nm to 2 μm;
softening a facial surface of an appliance substrate; and
pressing the facial surface into the abrasive particulate blend while the facial surface is in a softened state to form an abrasive matrix partially embedded in the facial surface.

23. The method of claim 22, wherein the abrasive crystals include aluminum oxide particles, magnesium oxide particles, sodium chloride particles, sodium bicarbonate particles, or diamond particles.

24. The method of claim 22, wherein the abrasive crystals have an average particle size from 40 μm to 180 μm, and the abrasive matrix has a grit count of 80 to 280.

25. The method of claim 22, wherein the metal-containing particles include gold, silver, copper, zinc, platinum, an alloy thereof, a salt thereof, or a combination thereof.

26. The method of claim 22, wherein the facial surface of the appliance substrate includes a solvent-softenable plastic material, and wherein softening is by chemical softening.

27. The method of claim 26, where chemical softening includes contacting the facial surface with a solvent selected from 1,2-dichloroethane, acetone, cyclohexanone, dichloromethane, methyl ethyl ketone (MEK), methyl benzene, tetrahydrofuran, or a combination thereof.

28. The method of claim 27, wherein the solvent-softenable plastic material selected from acrylic polymer, methacrylic polymer, acrylonitrile butadiene styrene, polyacetal, cellulose acetate butyrate, polyethylene, polycarbonate, polyethylene terephthalate, polyethylene terephthalate glycol, polypropylene, polystyrene, polyvinyl chloride, or combinations thereof.

29. The method of claim 22, wherein the facial surface of the appliance substrate includes a thermally-softenable plastic material, and wherein softening is by thermal softening.

30. The method of claim 29, wherein the heat-softenable plastic material is a thermoplastic selected from acrylic polymer, methacrylic polymer, acrylonitrile butadiene styrene, polyester, polyimide, polytetrafluoroethylene, nylon, polyacetal, cellulose acetate butyrate, polyethylene, polycarbonate, polyethylene terephthalate, polyethylene terephthalate glycol, polypropylene, polystyrene, polyvinyl chloride, or combinations thereof.

31. A method of rejuvenating a skin surface, comprising applying a machine-actuated abrasive matrix on a skin surface, wherein the abrasive matrix comprises abrasive crystals having an average particle size from 25 μm to about 250 μm and a plurality of metal-containing particles having an average particle size from 5 nm to 2 μm associated with surfaces of the abrasive crystals.

32. The method of claim 31, further comprising depositing a plurality of the metal-containing particles on the skin surface while the machine-actuated abrasive matrix is in contact with the skin surface.

33. The method of claim 31, wherein the abrasive crystals include aluminum oxide particles, magnesium oxide particles, sodium chloride particles, sodium bicarbonate particles, or diamond particles.

34. The method of claim 31, wherein the abrasive crystals have an average particle size from 40 μm to 180 μm, and the abrasive matrix has a grit count of 80 to 280.

35. The method of claim 31, wherein the metal-containing particles include gold, silver, copper, zinc, platinum, an alloy thereof, a salt thereof, or a combination thereof.

36. A method of therapeutically treating a skin surface, comprising abrading a skin surface with an abrasive matrix of an abrasive skin care appliance, wherein the comprises abrasive crystals having an average particle size from 25 μm to about 250 μm and a plurality of metal-containing particles having an average particle size from 5 nm to 2 μm associated with surfaces of the abrasive crystals.

37. The method of claim 36, wherein abrading the skin surface includes contacting the abrasive matrix with the skin surface while the abrasive matrix is rotating at from 2,000 RPM to 7,000 RPM.

38. The method of claim 36, wherein abrading the skin surface includes contacting the abrasive matrix with the skin surface while the abrasive matrix is vibrationally oscillating at from 2,000 VPM to 12,000 VPM.

39. The method of claim 36, wherein abrading the skin surface includes manually abrading the skin surface by contacting the abrasive matrix with the skin surface without the use of electrical power.
